(19) United States
(12) Patent Application Publication
(10) Pub. No.: US 2007/0179321 A1
(43) Pub. Date: Aug. 2, 2007

(54) PROCESS FOR THE PRODUCTION OF 4,4-DIPHENYLCYCLOHEXANOL

(76) Inventors: Ferdinand Koerner, Bergkamen (DE); Manfred Kirchhuebel, Unna (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/612,117

(22) Filed: Dec. 18, 2006

Related U.S. Application Data
(60) Provisional application No. 60/753,421, filed on Dec. 27, 2005.

(30) Foreign Application Priority Data
Dec. 19, 2005 (DE) 10 2005 061 634.8

(51) Int. Cl. C07C 37/01; C07F 9/02

(52) U.S. Cl. 568/17; 568/809

(57) ABSTRACT

A new process for tie production of 4,4-diphenylcyclohexanol is described.

This application claims the benefit of the filing date of U.S. Provisional Application Ser. No. 60/753,421, filed Dec. 27, 2005.

The invention relates to the subject that is characterized in the claims, i.e., a new process for the production of 4,4-diphenylcyclohexanol.

The use of contrast media for the visualization of the intravascular space (blood pool imaging) is one of the important applications in MRI angiography. In this connection, one compound in particular, the above-mentioned MS-325 (U.S. Pat. No. 6,676,929 and WO 96/23526) that was approved by the EMEA under the name Vasovist®, has proven its value. The synthesis of MS-325 is described in Synthetic Communications, 26(13), 2511-2522 (1996) and Synthetic Communications, 29(14), 2377-2391 (1999).

Within the scope of the development of this compound, it was desired to provide larger amounts of substances. Since tie substance is administered to humans, strict requirements on the purity of the end product as well as on the intermediate products must be imposed. Because of the many uses that are to be expected, it should also be possible to produce such a high-grade product at representative cost in terms of price and time. There is therefore a desire to have as economically advantageous a synthesis as possible.

A very important intermediate product of the synthesis of MS-325 is the 4,4-diphenylcyclohexanol (I)

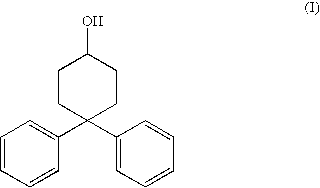

The processes, previously disclosed in the prior art, for the production of 4,4-diphenylcyclohexanol (I) from 4,4-diphenylcyclohex-2-enone (II) 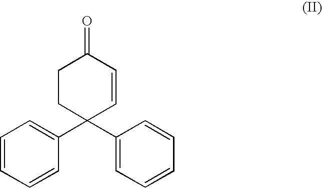
were always in two stages, whereby first the double bond is hydrogenated heterogenically and catalytically, preferably with use of a palladium catalyst (Amedio et al., Synt. Comm. 28(20) 1998, 3895-3906, Freeman et al., J. Org. Chem. 54(4) 1999, 782-789). After the 4,4-diphenylcyclohexanone that is formed is isolated, the reduction of the keto function with complex metal hydrides, for example sodium borohydride or lithium aluminum hydride, is then carried out in a second step.

With respect to the raw material and disposal costs as well as throughput time, however, a two-stage reaction scheme is disadvantageous. In addition, the most economical reducing agents (possibly hydrogen) are not used, which results in a further increase in production costs.

A direct, one-stage hydrogenation was mentioned in the available literature, only in U.S. Pat. No. DE 1,793,611, Example No. 8. There, the described reaction with use of platinum oxide was used for the production of 4,4-diphenylcyclohexanol. A more precise indication of the reaction conditions is lacking. The 4,4-diphenylcylcohexanol that was formed was further reacted without characterization; the total yield over 2 stages was only 41% by use of 4,4-diphenylcyclohex-2-enone.

There is therefor a need for a new process for the synthesis of 4,4-diphenylcyclohexanol from 4,4-diphenylcyclohex-2-enone, which is both economical and requires as little tine as possible.

The process according to the invention fulfills the imposed requirements to a large extent.

The invention relates to a process for the production of the compound 4,4-diphenylcyclohexanol of formula (I) 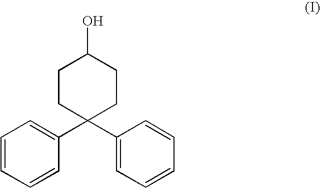
characterized in that the compound 4,4-diphenylcyclohex-2-enone of formula (II) 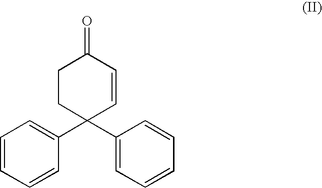
is dissolved in an organic solvent and then is hydrogenated in the presence of a nickel catalyst with hydrogen, whereby the reaction time is at least 1 minute, and the reaction temperature is 0° C. to 100° C., and the 4,4-diphenylcyclohexanol that is obtained is optionally purified.

The solvent is preferably selected from isopropanol, methanol, ethanol, n-propanol, n-butanol, THF, acetone, ethyl acetate, dichloromethane, toluene or mixtures

In an especially preferred embodiment, the solvent THF is contained; the solvent in particular is a mixture of THF/methanol in the ratio of 1:10 to 10:1.

The use of pure THF has a solvent (i.e., no further solvents are present in the reaction batch) is quite especially preferred.

Pure THF is defined as THF with a purity of at least 99% by weight.

A solvent is defined as a liquid in which 4,4-diphenylcyclohexanol is completely soluble at at least a temperature of between 0° C. and 100° C. at a concentration of at least 1% by weight, preferably at least 5% by weight, in particular at least 10% by weight.

In a preferred embodiment, 4,4-diphenylcyclohex-2-enone is mixed with the organic solvent at a ratio of 1:100 to 10:1, in particular at a ratio of 1:50 to 1:1 , quite especially preferably at a ratio of 1:20 to 1.3.

In a quite especially preferred embodiment, 4,4-diphenylcyclohex-2-enone is mixed with THF at a ratio of 1:3 to 1:20.

The hydrogenation is carried out in the presence of the nickel catalyst, preferably Raney nickel.

The purification is carried out according to the methods that are known to one skilled in the art; the reaction mixture is preferably filtered and concentrated by evaporation, and a solvent, preferably methanol, is added at a ratio of 1:10 to 10:1, and the solvent is distilled off, and the 4,4-diphenylcyclohexanol is isolated by filtration,

The reaction time is preferably at least 5 minutes, especially preferably at least 10 minutes, most preferably at least 20 minutes. The length of time to carry out the reaction can be arbitrary; for economic reasons, however, the reaction time usually does not exceed 48 hours.

The reaction temperature is preferably 10° C. to 80° C., especially preferably between 15° C. and 50° C., quite especially preferably between 20° C. and 40° C.,

The thus obtained 4,4-diphenylcyclohexanol can then be used for synthesis of Vasovist in its formulated form, for synthesis of Gadofosveset or one or its salts, in particular sodium salt, or for synthesis of Fosveset, as described in, for example, U.S. Pat. No. 6,676,929 or U.S. Pat. No. 5,919,967.

The starting substance 4,4-diphenylcyclohex-2-enone is generally known to one skilled in the art and can be synthesized, e.g., as described in Amedio et al., Synt. Comm. 28(20) 1998, 3895-3906, Zimmermann et al. J. Am. Chem. Soc. 84, 1962, 4527 or Bordwell et al. J. Org. Chem. 28, 1963, 2544.

In addition, the invention relates to a process for the production of MS-325, characterized by the steps
a) Reaction of the compound 4,4-diphenylcyclohex-2-enone of formula (II) 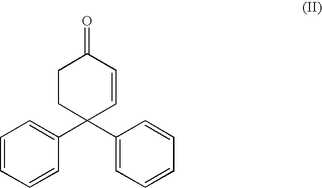
   is dissolved in an organic solvent and then
   is hydrogenated with hydrogen in the presence of a nickel catalyst,
   whereby the reaction time is at least 1 minute,
   and the reaction temperature is 0° C. to 100° C.,
   to form 4.4-diphenylcyclohexanol
b) Optional purification of 4,4-diphenylcyclohexanol
c) Reaction of4,4-diphenylcyclohexanol with PCl₃ and then imidazole in THF to form bis(amino)phosphino reaction product, and
d) Reaction of the reaction product that is obtained in c) to form Fosveset
e) Reaction of Fosveset and Gd₂O₃ to form Gadofosveset (MS-325).

Steps b) to e) are described in U.S. Pat. No. 5,919967, and reference is expressly made herewith to the disclosure of the patent, and the content of the patent is incorporated in this application to the extent in which it relates to the production of MS-325 starting from 4,4-diphenylcyclohexanol.

In another embodiment, this invention relates to a process for the production of an MS-325-containing preparation that is suitable for diagnosis by means of MRI, characterized in that first MS-325 is produced according to the above-cited process, and then it is brought into a form that is acceptable for diagnostic application in humans with adjuvants and additives that are commonly used in galenicals.

In an especially preferred embodiment, the preparation is suitable for the i.v. application, and is in particular a preparation that can be obtained under the trade name Vasovist®.

The advantages of the new process are:
1. Considerably higher total yield relative to the prior art (see below)
2. Simple procedure (one-stage reduction)
3. Reasonably-priced hydrogenating agent (hydrogen)
4. High purity of the 4,4-diphenylcyclohexanol that is obtained

### EXAMPLES

### Example 1

100 g of 4,4-diphenylcyclohex-2-enone is dissolved in 750 ml of tetrahydrofuran. 10 ml of an aqueous Raney nickel suspension, washed free of salt in advance, is added. While being stirred, the suspension is hydrogenated at a hydrogen pressure of 5 bar and a temperature of 25 to 30° C. for 60 minutes. Nickel catalyst is filtered out, and the filtrate is concentrated by evaporation under normal pressure up to a temperature of 80 to 85° C. Then, 500 ml of methanol is added, concentrated by evaporation to a residual volume of 200 ml and cooled to 0° C. The crystallizate is isolated, washed with methanol and dried.

Yield: 95 g (corresponds to 93% of theory)

Content: 99.7% surface area (HPLC).

### Example 2

80.0 g of 4,4-diphenylcyclohex-2-enone is dissolved in 750 ml of tetrahydrofuran/methanol mixture 7:3 (v/v). 10 ml of an aqueous Raney niekel suspension that is washed free of salt in advance is added. While being stirred, the suspension is hydrogenated at a hydrogen pressure of 5 bar and a temperature of 25 to 30° C. for 60 minutes. Nickel catalyst is filtered out, and the filtrate is concentrated by evaporation under vacuum up to a temperature of 40 to 45° C. Then, 400 ml of methanol is added, anti 250 ml of solvent is distilled off under vacuum. It is cooled to 0° C., the crystallizate is isolated, washed with methanol and dried.

Yield: 68.5 g (corresponds to 84% of theory).

Content: 99.7% surface area (HPLC).

### Example 3

80.0 g of 4,4-diphenylcyclohex-2-enone is suspended in 750 ml of isopropanol. 10 ml of an aqueous Raney nickel suspension that is washed free of salt in advance is added. While being stirred, the suspension is heated at a hydrogen pressure of 5 bar up to a temperature of 70° C. and hydrogenated for 120 minutes. Nickel catalyst is filtered out in a hot state, the filtrate is concentrated by evaporation under vacuum up to a volume of 300 ml, and it is cooled to 0° C. The crystallizate is isolated, washed with isopropanol and dried.

Yield: 66.0 g (corresponds to 81% of theory).

Content: 99.1% surface area (HPLC).

### Example 4

80.0 g of 4,4-diphenylcyclohex-2-enone is suspended in 750 ml of methanol. 10 ml of an aqueous Raney nickel suspension that is washed free of salt in advance is added. While being stirred, the suspension is heated at a hydrogen pressure of 5 bar up to a temperature of 70° C. and hydrogenated for 90 minutes. Nickel catalyst is filtered out in a hot state, and the filtrate is concentrated by evaporation under vacuum up to a volume of about 150 ml. Then, it is cooled to 0° C., the crystallizate is isolated, washed with methanol and dried.

Yield: 72 g (corresponds to 89% of theory).

Content: 73.8% surface area of 4,4-diphenylcyclohexanol (HPLC) 25.4% surface area of 4,4-diphenylcyclohex-2-enone (HPLC).

### Example 5 (Comparison Test)

57.0 g of 4,4-diphenylcyclohex-2-enone is dissolved in 1000 ml of ethanol.

1.0 g of palladium on an activated carbon carrier (palladium content of 5 to 10% by weight) is added. While being stirred, the suspension is heated at a hydrogen pressure of 5 bar up to a temperature of 80° C., and it is hydrogenated for 75 minutes. By means of TLC monitoring of the hydrogenating suspension no formation of 4,4-diphenylcyclohexanol can be determined.

1.0 g of palladium on an activated carbon carrier (palladium content of 5 to 10% by weight) is again added. While being stirred, the suspension is heated at a hydrogen pressure of 6.8 bar up to a temperature of 100° C., and it is hydrogenated for 60 minutes. By means of TLC monitoring of the hydrogenating suspension, the formation of about 10 to 20% of 4,4-diphenylcyclohexanol can be determined. 1.0 g of platinum on an activated carbon carrier (platinum content about 10% by weight) is added. While being stirred, the suspension is heated at a hydrogen pressure of 7.0 bar up to a temperature of 100° C., and it is hydrogenated for 60 minutes. By means of TLC monitoring of the hydrogenating suspension, the formation of 50% 4,4-diphenylcyclohexanol can be determined .

Catalyst is filtered out, and the filtrate is concentrated by evaporation up to a volume of about 100 ml. Then, 250 ml of water is added at a temperature of 55 to 60° C. and cooled to 10° C. The crystallizate is isolated, washed with water, and dried.

Yield: 56.0 g (corresponds to 96% of theory)

Content: 50% 4,4-diphenylcylcohexanol as well as 50% sum that consists of 4,4-diphenylcylcohex-2-enone and 4,4-diphenylcyclohexanone (determined by TLC).

### Example 6 (Comparison Test)

55.0 g of reaction product from Example 5 is dissolved in 1000 ml of tetrahydrofuran.

3.0 g of platinum on an activated carbon carrier (platinum content of about 10% by weight) is added. While being stirred, the suspension is hydrogenated at a hydrogen pressure of 5 bar at a temperature of 20 to 25° C. for 60 minutes. By means of TLC monitoring of the hydrogenating suspension, no change relative to the charging material can be determined.

Again, 2.0 g of platinum on an activated carbon carrier (platinum content about 10% by weight) is added. While being stirred, the suspension is hydrogenated at a hydrogen pressure of 5 bar at a temperature of 25 to 30° C. for 60 minutes. By means of TLC monitoring of the hydrogenating suspension, no change relative to the charging material can be determined.

While being stirred, the suspension is hydrogenated at a hydrogen pressure of 6.6 bar at a temperature of 80° C. for 30 minutes. By means of TLC monitoring of the hydrogenating suspension, the formation of nonpolar secondary compounds on the order of magnitude of 30 to 50% can be determined.

Without further elaboration it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2005 061 634.8, filed Dec. 19, 2005, and U.S. Provisional Application Ser. No. 60/753,421, filed Dec. 19, 2005, are incorporated by reference Therein.

The preceding examples can be repeated with similar success by substituting the generically; or specifically described reactants and/or operating conditions of this invention for those used in the preceding examples.

From the foregoing description, one skilled in the art can easily ascertain the essential characteristics of this invention and, without departing from the spirit and scope thereof, can male various changes and modifications of the invention to adapt it to various usages and conditions.

1. Process for the production of 4,4-diphenylcyclohexanol (I) 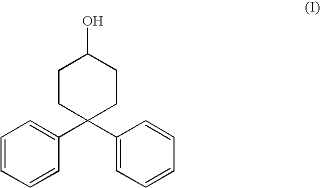
characterized in that the compound of formula (II) 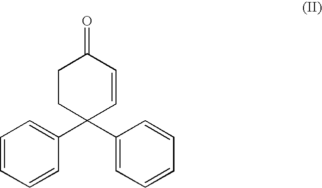
is dissolved in an organic solvent and then is hydrogenated in the presence of a nickel catalyst with hydrogen, whereby the reaction time is at least 1 minute, and the reaction temperature is 0° C. to 100° C., and the 4,4-diphenylcyclohexanol that is obtained is optionally purified.

2. Process for the production of MS-325, characterized by the following steps:
a) Executing the process according to claim 1,
b) Optional purification of 4,4-diphenylcyclohexanol
c) Reaction of 4,4-diphenylcyclohexanol with PCl₃ and then imidazole in THF to form the bis(amino)phosphino reaction product, and
d) Reaction of the reaction product that is obtained in c) to form Fosveset
e) Reaction of Fosveset and Gd₂O₃ to form Gadofosveset (MS-325).

3. Process according to claim 1, wherein the organic solvent is
isopropanol, methanol, ethanol, n-propanol, n-butanol, THF, acetone, ethyl acetate, dichloromethane, toluene or mixtures thereof.

4. Process according to claim 3, wherein the organic solvent contains THF.

5. Process according to claim 4, wherein the organic solvent is THF, and no additional solvents are present in the reaction batch.

6. Process according to claim 1, wherein the catalyst is Raney nickel.

7. Process according to claim 1, wherein the reaction temperature is between 10° C. and 80° C.

8. Process according to claim 1, wherein the reaction time is at least 10 minutes.
